SEQUENCE LISTING (34 sequences)

SEQ ID NO: 1 - protein, 347 aa, Homo sapiens [sequence omitted]

SEQ ID NO: 2 - protein, 967 aa, Homo sapiens
MAKGFYISKSLGILGILLGVAAVCTIIALSVVYSQEKNKNANSSPVASTTPSASATTNPASATTLDQSKAWNRYRLPNTLKPDSYQVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 3 - protein, 9 aa, Homo sapiens
RGPLQLERL

SEQ ID NO: 4 - protein, 11 aa, Homo sapiens
KGQTLLAVAKS

SEQ ID NO: 5 - protein, 12 aa, Homo sapiens
RVAAGAFQGLRQ

SEQ ID NO: 6 - protein, 13 aa, Homo sapiens
KALGHLDLSGNRL

SEQ ID NO: 7 - protein, 12 aa, Homo sapiens
KDLLLPQPDLRY

SEQ ID NO: 8 - protein, 15 aa, Homo sapiens
RLHLEGNKLQVLGKD

SEQ ID NO: 9 - protein, 18 aa, Homo sapiens
KENQLEVLEVSWLHGLKA

SEQ ID NO: 10 - protein, 20 aa, Homo sapiens
RTLDLGENQLETLPPDLLRG

SEQ ID NO: 11 - protein, 25 aa, Homo sapiens
RNALTGLPSGLFQASATLDTLVLKE

SEQ ID NO: 12 - protein, 28 aa, Homo sapiens
KLQELHLSSNGLESLSPEFLRPVPQLRV

SEQ ID NO: 13 - protein, 11 aa, Homo sapiens
RSIQLPTTVRD

SEQ ID NO: 14 - protein, 12 aa, Homo sapiens
RDHSAIPVINRA

SEQ ID NO: 15 - protein, 14 aa, Homo sapiens
RGVGGSQPPDIDKT

SEQ ID NO: 16 - protein, 13 aa, Homo sapiens
KDLTALSNMLPKG

SEQ ID NO: 17 - protein, 11 aa, Homo sapiens
RVNYDEENWRK

SEQ ID NO: 18 - protein, 13 aa, Homo sapiens
KEATDVIIIHSKK

SEQ ID NO: 19 - protein, 12 aa, Homo sapiens
KDLMVLNDVYRV

SEQ ID NO: 20 - protein, 14 aa, Homo sapiens
KDNEETGFGSGTRA

SEQ ID NO: 21 - protein, 15 aa, Homo sapiens
KVVATTQMQAADARK

SEQ ID NO: 22 - protein, 14 aa, Homo sapiens
RMLSSFLSEDVFKQ

SEQ ID NO: 23 - protein, 13 aa, Homo sapiens
RQQQDYWLIDVRA

SEQ ID NO: 24 - protein, 14 aa, Homo sapiens
RYLSYTLNPDLIRK

SEQ ID NO: 25 - protein, 13 aa, Homo sapiens
KEVVLQWFTENSK

SEQ ID NO: 26 - protein, 17 aa, Homo sapiens
RAQIINDAFNLASAHKV

SEQ ID NO: 27 - protein, 18 aa, Homo sapiens
RENSLLFDPLSSSSSNKE

SEQ ID NO: 28 - protein, 17 aa, Homo sapiens
KTELVEPTEYLVVHLKG

SEQ ID NO: 29 - protein, 16 aa, Homo sapiens
RFSTEYELQQLEQFKK

SEQ ID NO: 30 - protein, 18 aa, Homo sapiens
RQYMPWEAALSSLSYFKL

SEQ ID NO: 31 - protein, 17 aa, Homo sapiens
RRFSTEYELQQLEQFKK

SEQ ID NO: 32 - protein, 23 aa, Homo sapiens
KLFNDYGGGSFSFSNLIQAVTRR

SEQ ID NO: 33 - protein, 25 aa, Homo sapiens
KDSQYEMDSEFEGELADDLAGFYRS

SEQ ID NO: 34 - protein, 31 aa, Homo sapiens
KQDATSTIISITNNVIGQGLVWDFVQSNWKK

(19) United States
(12) Patent Application Publication
(10) Pub. No.: US 2013/0102011 A1
(43) Pub. Date: Apr. 25, 2013

(54) HUMAN LEUCINE-RICH A-2-GLYCOPROTEIN-1 AND AMINOPEPTIDASE N AS RISK INDICATORS FOR CANCER

(71) Applicants: Aimin Zhou, Solon, OH (US); Hongli Liu, Xi-an (CN); Chun Zeng, Cleveland, OH (US)

(72) Inventors: Aimin Zhou, Solon, OH (US); Hongli Liu, Xi-an (CN); Chun Zeng, Cleveland, OH (US)

(73) Assignee: CLEVELAND STATE UNIVERSITY, Cleveland, OH (US)

(21) Appl. No.: 13/618,005

(22) Filed: Sep. 14, 2012

Related U.S. Application Data
(60) Provisional application No. 61/534,548, filed on Sep. 14, 2011.

(51) Int. Cl. G01N 33/68

(52) U.S. Cl. CPC G01N 33/6893
USPC 435/7.23; 435/7.92; 435/23

(57) ABSTRACT

Diagnostic tests for characterizing a test subject's risk of developing or having cancer, based on determining the level of LRG1 and/or CD13 in a bodily sample obtained from a test subject are described. Levels of LRG1 and/or CD13 are then compared to a predetermined value that is derived from measurements of the levels of LRG1 and/or CD13 in comparable bodily samples obtained from control subjects. Such comparison characterizes the test subject's risk of developing or having cancer.

### CROSS-REFERENCE TO RELATED APPLICATIONS

This application claims priority to U.S. Provisional Patent Application No. 61/534,548, filed Sep. 14, 2011, which is incorporated herein by reference in its entirety.

### BACKGROUND

The present disclosure relates to the field of cancer treatment. More specifically, the disclosure relates to a diagnostic test which may be used to determine whether an individual or test subject is at risk of developing or having cancer.

Cancer is a family of diseases that result from uncontrolled growth and the spread of abnormal cells. Uncontrolled spreading of cancerous cells leads to morbidity and mortality. The success rate of therapeutic treatment of cancer is usually greatly improved when the disease is diagnosed at an early stage. In general, cancer diagnosis is based upon symptoms, the results of physical examination, and screening tests for validated clinical indicators (biomarkers) in bodily fluids. In addition, multiple techniques including, mammograms, colonoscopy, and computed tomography are routinely used for early cancer diagnosis. However, these diagnostic methods are almost all site-specific, invasive, and expensive. In recent years, noninvasive tests that detect metabolites and excreted molecules in bodily fluids have exhibited potential as simple, cheap, and reliable methods for cancer diagnosis. Goessel, C., Curr. Med. Chem., Vol. 10, No. 8, pp. 691-706 (2003). For example, cytology, growth factors, and other biomolecules presented in sputum samples are used for the diagnosis of lung cancer; a number of genetic and epigenetic protein markers in stool have been identified for detecting colorectal cancer; and metabolites and macromolecules excreted in urine exhibit promise as targets for prostate, renal, and bladder cancers.

Unmodified human leucine-rich α-2-glycoprotein-1 (LRG1) is a serum glycoprotein that is 312 amino acids in length, 66 of which are leucine, and has a predicted molecular weight of 34 to 36 kDa. The secreted form of LRG1 contains one attached galactosamine-based and four attached glucosamine-based oligosaccharides, and has a molecular weight ranging between 44 to 55 kDa. The LRG1-modified glycoprotein contains two intrachain disulfide bonds, and has a normal plasma concentration of 21-50 μg/ml. The function of LRG1 remains largely unknown, but it is possible that LRG1 may play a role in cell adhesion due to the leucine-rich repeats, and the impact it has upon granulocytic differentiation and expression in neutrophils. Takahashi et al., Proc. Natl. Acad. Sci. USA, (1985), Vol. 82, No. 7, pp. 1906-1910. LRG1 may be involved in the TGF-βR II signaling pathway. The binding of serum LRG1 to cytochrome c suggests that the protein may also play a role in cell survival and apoptosis. Recently, LRG1 was found to be upregulated in sera and tumors of ovarian cancer patients. Anderson et al., J. Ovarian Res. Vol. 3, pp. 21 (2010).

Aminopeptidase N (APN or CD13), a Zn²⁺-dependent ectopeptidase, is a transmembrane protein that cleaves N-terminal neutral amino acids of various peptides and proteins. Although CD13 is widely expressed in monocytes, macrophages, and myeloid cells, expression of CD13 has also been found in non-hematopoietic cells such as fibroblasts, brain cells, and epithelial cells of the liver, kidney, and intestine. Aminopeptidase N is known to have a variety of biological functions. For example, CD13 is involved in both inflammatory and immunological responses, signal transduction, antigen processing, and neuropeptide and cytokine degradation. High expression of CD13 has been found to correlate with increased malignancy of cancer cells. Wickström et al., Cancer Sci., Vol. 102, No. 3, 501-508 ((2011). Furthermore, studies have shown that CD13 contributes to cancer cell proliferation, tumor invasion, and angiogenesis. Fukusawa, et al., Cancer Lett., Vol. 243, No. 1, pp. 135-143 (2006).

Hepatocellular carcinoma (HCC) or malignant hepatoma is the most common type of liver cancer. Most cases of HCC are secondary to either a viral hepatitis infection, such as hepatitis B or C, or cirrhosis. Interestingly, nephrotoxicity and hepatotoxicity result in increased excretion of CD13 in urine, which can be used to evaluate kidney damage and function. Although hepatocellular carcinoma is quite rare in the United States, it accounts for most liver cancers. HCC is more common in men than women, and the highest prevalence of HCC is found in parts of Africa and Asia. In countries where hepatitis is not endemic most malignant cancers in the liver are not primary HCC but metastases of cancer from elsewhere in the body, typically, the colon. Treatment options of HCC and prognosis are dependent on many factors but especially on tumor size and staging. High-grade tumors usually have a poor prognosis, while low-grade tumors may go unnoticed for many years.

Aggressive surgery or a liver transplant may successfully treat small or slow-growing tumors if they are diagnosed early. However, few patients are diagnosed early. Chemotherapy and radiation treatments are not usually effective, but may be used to shrink large tumors in an effort to increase surgical success. Sorafenib tosylate, an oral medicine that blocks tumor growth, has been approved for patients with advanced hepatocellular carcinoma.

HCC prognosis is usually poor, because only 10-20% of hepatocellular carcinomas can be removed completely using surgery. If hepatocellular carcinomas cannot be completely removed by surgical methods, fatality usually results within 3-6 months.

Accordingly, the need still exists for additional diagnostic tests for characterizing an individual's risk of developing or of having cancer. Diagnostic tests which evaluate validated clinical indicators independently of traditional cancer screening methods and risk factors, such as alcoholism in the case of HCC, are especially desirable.

### SUMMARY

The present disclosure provides new diagnostic tests for characterizing an individual's risk of developing or having cancer. The present tests may be useful for identifying those individuals who are in need of highly aggressive cancer therapies as well as those individuals who require no therapies targeted at preventing cancer, especially those of the bladder, prostate, urinary tract, breast, the ovaries, leukemia, lymphoma, myeloma, stomach, pancreas, colon, renal, and those of the liver. Use of the present diagnostic tests can be prompted by the discovery of symptoms of cancer and the results of physical examination. Present diagnostic tests are often site-specific, invasive, and expensive. For example, diagnostic tests for liver cancer include an α-fetoprotein blood test, or diagnostic tests including, triphasic helical CT scans, magnetic resonance cholangiopancreatography, ultrasound, and cytology. Thus, the present diagnostic tests, which involve assessing levels of LRG1, CD13, or the combination thereof in a urine or serum sample from a test subject, may provide additive predictive value beyond that seen with clinical and diagnostic risk factors currently employed by physicians.

In one embodiment, the present diagnostic test comprises determining the level of LRG1, CD13, or the combination thereof in a sample of bodily fluid obtained from the individual or test subject. In one embodiment, the bodily fluid sample is urine. In another embodiment, the level of LRG1, CD13, or the combination thereof in the sample from the test subject is compared to predetermined values that are derived from measurements of LRG1, CD13, or the combination thereof in comparable samples obtained from the general population or a select population of human subjects. Such comparison may characterize the test subject's risk of developing cancer.

For example, test subjects whose urine levels of LRG1, CD13, or both are higher than the predetermined values are at greater risk of developing or having HCC than individuals whose urine LRG1, CD13, or both levels are at or lower than the predetermined value. Moreover, the extent of the difference between the test subjects LRG1, CD13, or both levels and the predetermined value is also useful for characterizing the extent of the risk and thereby, determining which individuals would most greatly benefit from certain therapies.

In one embodiment, the diagnostic test comprises determining the level of one or more select LRG1 metabolites, CD13 metabolites, or both types of metabolites in a bodily sample obtained from the test subject.

In one embodiment, the present invention provides a method for evaluating therapy in a subject suspected of having or having cancer. In another embodiment, the method comprises determining the levels of one or more of the present risk factors, including a LRG1 level, a CD13 level, or a combination thereof, in a bodily fluid sample taken from the subject prior to therapy and a corresponding bodily fluid sample taken from the subject during or following therapy. A decrease in the level of the selected risk factor in the sample taken after or during therapy as compared to the level of the selected risk factor in the sample taken before therapy is indicative of a positive effect of the therapy on cancer in the treated subject.

### BRIEF DESCRIPTION OF THE FIGURES

The accompanying drawings, which are incorporated in and constitute a part of this specification, illustrate some embodiments of the inventions, and together with the description, serve to explain principles of the inventions.

FIG. 1 provides a scheme showing the steps involved in one embodiment of the diagnostic tests, involving sample collection, protein separation, protein digestion, mass spectrometry, data analysis, and further biological analysis.

FIG. 2 shows the amino acid sequence for human leucine-rich α-2-glycoprotein 1 (SEQ ID NO: 1; LRG1). Matched peptides shown in bold.

FIG. 3 shows the amino acid sequence for human membrane aminopeptidase N (SEQ ID NO: 2; CD13). Matched peptides shown in bold.

FIG. 4 shows urinary excretion of LRG1 in healthy subjects. M represents protein markers. N005-N040 represent the protein levels associated with healthy control subjects. A108 represents the protein levels associated with a subject who is positive for HCC.

FIG. 5 shows urinary excretion of CD13 in healthy subjects. M represents protein markers. N001-N040 represent the protein levels associated with healthy control subjects. A108 represents the protein levels associated with a subject who is positive for HCC.

FIG. 6 urinary excretion of CD13 and LRG1 in healthy subjects. M represents protein markers. N041-N048 represent the protein levels associated with the healthy control subjects. A108 represents the protein levels associated with a subject who is positive for HCC. Analysis was completed using immunoblot assays known to one of ordinary skill in the art.

FIG. 7 shows urinary excretion of CD13 and LRG1 in HCC subjects. M represents protein markers. N represent the protein levels associated with a healthy subject. Numbers represent HCC patient samples. Analysis was completed using immunoblot assays known to one of ordinary skill in the art.

FIG. 8 provides a picture showing the results of a western blot analysis carried out to determine the stability of LRG1 in urine.

FIG. 9 shows a probability based Mowse score. Ions score −10*Log(P), where P is the probability that the observed match is a random event. Individual ions scores >49 indicate identity or extensive homology (p<0.05). Protein scores are derived from ions scores as a non-probabilistic basis for ranking protein hits. LRG1 has a probability based Mowse score of approximately 380.

FIG. 10 shows sequence match results for the comparison of MS/MS fragments from patients to the amino acid sequence of human leucine-rich α-2-glycoprotein 1 (LRG1).

FIG. 11 shows a probability based Mowse score. Ions score −10*Log(P), where P is the probability that the observed match is a random event. Individual ions scores >48 indicate identity or extensive homology (p<0.05). Protein scores are derived from ions scores as a non-probabilistic basis for ranking protein hits. CD13 has a probability based Mowse score of approximately 825.

FIG. 12 shows sequence match results for the comparison of MS/MS fragments from patients to the amino acid sequence of human membrane aminopeptidase N (CD13).

FIG. 13 provides a graph showing the quantitative analysis of LRG1 in urine by ELISA. The graph includes results for a control, patients with chronic hepatitis B (CHB), liver cirrhosis (LC), hepatocellular carcinoma (HCC) and patients who have had their HCC tumors removed (HCC-treated).

### DETAILED DESCRIPTION

The present inventions will now be described by reference to some more detailed embodiments, with occasional reference to the accompanying drawings. These inventions may, however, be embodied in different forms and should not be construed as limited to the embodiments set forth herein. Rather, these embodiments are provided so that this disclosure will be thorough and complete, and will fully convey the scope of the inventions to those skilled in the art.

Unless otherwise defined, all technical and scientific terms used herein have the same meaning as commonly understood by one of ordinary skill in the art to which these inventions belong. The terminology used in the description of the inventions herein is for describing particular embodiments only and is not intended to be limiting of the inventions. As used in the description of the inventions and the appended claims, the singular forms “a,” “an,” and “the” are intended to include the plural forms as well, unless the context clearly indicates otherwise. All publications, patent applications, patents, and other references mentioned herein are incorporated by reference in their entirety.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present inventions. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the inventions are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

As used herein, the term “diagnosis” can encompass determining the nature of disease in a subject, as well as determining the severity and probable outcome of disease or episode of disease and/or prospect of recovery (prognosis). “Diagnosis” can also encompass diagnosis in the context of rational therapy, in which the diagnosis guides therapy, including initial selection of therapy, modification of therapy (e.g., adjustment of dose and/or dosage regimen), and the like.

The terms “individual,” “host,” “subject,” and “patient” are used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Diagnosis of humans is of particular interest.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

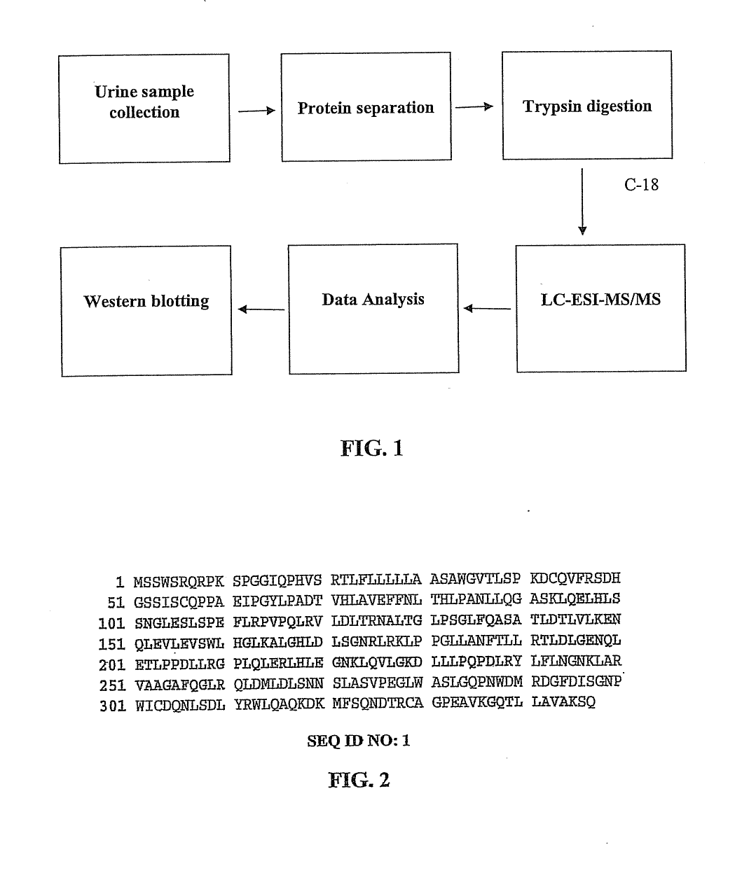

In one aspect, a method of characterizing a subject's risk of having cancer is provided. The method includes the steps of: (a) determining a level of LRG1 and/or CD13 or one or more metabolites thereof in a biological sample obtained from the subject by analyzing the biological sample using a protein analytic method; and (b) comparing the determined level of the LRG1 and/or CD13 or one or more metabolites thereof in the biological sample to at least one predetermined value; wherein a subject that has an elevated level of at least one of the LRG1 and/or CD13 or one or more metabolites thereof in comparison with one or more of the predetermined values is at risk of having cancer. At risk of having cancer, as defined herein, refers to an increased risk of having cancer in comparison with subjects who do not appear to have cancer. For example, the presence of LRG1 can indicate that a subject is 50% more likely to have cancer than a representative sample of subjects who do not exhibit increased LRG1 levels. In another embodiment, the comparison characterizes the subject's risk of developing cancer in the future. A scheme representing the steps in an embodiment of this method are shown in FIG. 1.

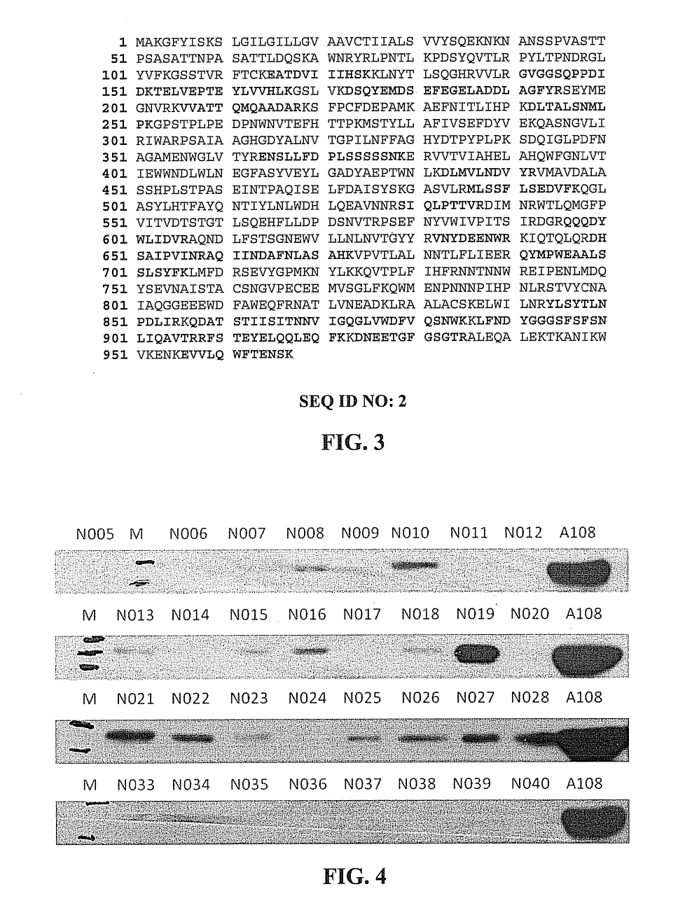

The method of cancer diagnosis described herein includes determining the levels of one or more of LRG1, CD13, or metabolites thereof in a biological sample. As noted herein, human leucine-rich α-2-glycoprotein-1 (LRG1, GeneBank accession number: NP_{—}443204) is a serum glycoprotein that is 312 amino acids in length. The amino acid sequence for LRG1 (SEQ ID NO: 1) is shown in FIG. 2. Some embodiments of the method of cancer diagnosis involve determining the level of LRG1 in a biological sample. Other embodiments of the method involve determining the level of CD13 in a biological sample. CD13 (Aminopeptidase N, GeneBank accession number: NP_{—}001141), a Zn²⁺-dependent ectopeptidase. The amino acid sequence for CD13 (SEQ ID NO: 2) is shown in FIG. 3.

The method of cancer diagnosis also includes determination of the levels of LRG1 metabolites and/or CD13 metabolites. Metabolites, as used herein, refers to recognizable products of protein catabolism. The body uses various known procedures to break down proteins, such as hydrolysis, and this protein catabolism can result in fragments of LRG1 and CD13 that are still recognizable as metabolites of these proteins, based on their amino acid sequence. Not includes herein as protein metabolites are those products of proteins such as urea and such which have been broken down to an extent that their source is no longer recognizable. Whether or metabolite is a recognizable product can be readily determined by one skilled in the art.

The method of cancer diagnosis can be used to diagnose a variety of different types of cancer. Cancer is a disease of abnormal and excessive cell proliferation. Cancer is generally initiated by an environmental insult or error in replication that allows a small fraction of cells to escape the normal controls on proliferation and increase their number. Cells that have developed growth advantages but have not yet become fully cancerous are referred to as precancerous cells. Cancer results in an increased number of cancer cells in a subject. These cells may form an abnormal mass of cells called a tumor, the cells of which are referred to as tumor cells. Tumors can be either benign or malignant. A benign tumor contains cells that are proliferating but remain at a specific site. The cells of a malignant tumor, on the other hand, can invade and destroy nearby tissue and spread to other parts of the body through a process referred to as metastasis.

Cancer is generally named based on its tissue of origin. There are several main types of cancer. Carcinoma is cancer that begins in the skin or in tissues that line or cover internal organs. Sarcoma is cancer that begins in bone, cartilage, fat, muscle, blood vessels, or other connective or supportive tissue. Leukemia is cancer that starts in blood-forming tissue such as the bone marrow, and causes large numbers of abnormal blood cells to be produced and enter the bloodstream. Lymphoma and multiple myeloma are cancers that begin in the cells of the immune system. The diagnostic methods described herein are suitable for identifying subjects who have cancers of the bladder, prostate, urinary tract, ovaries, kidneys, the liver, leukemia, myeloma, lymphoma, colon, stomach, pancreas and breast. More particularly, the method is suitable for diagnosing cancer of the liver, bladder, prostate, urinary tract, ovaries, or kidney. In particular, the methods are suitable for identifying a subject who has or is at an increased risk of developing liver cancer, or more specifically hepatocellular carcinoma.

### Biological Samples

Biological samples include, but are not necessarily limited to bodily fluids such as urine and blood-related samples (e.g., whole blood, serum, plasma, and other blood-derived samples), urine, cerebral spinal fluid, bronchoalveolar lavage, and the like. A preferred biological sample for diagnosis of liver cancer (e.g., HCC) is urine. Another example of a biological sample is a tissue sample. The levels of LRG1, CD13, or one or more metabolites thereof can be assessed either quantitatively or qualitatively, usually quantitatively. The levels of LRG1, CD13, or one or more metabolites thereof can be determined either in vivo or in vitro.

A biological sample may be fresh or stored (e.g. blood or blood fraction stored in a blood bank). Samples can be stored for varying amounts of time, such as being stored for an hour, a day, a week, a month, or more than a month. The biological sample may be a bodily fluid expressly obtained for the assays of this invention or a bodily fluid obtained for another purpose which can be subsampled for the assays of this invention.

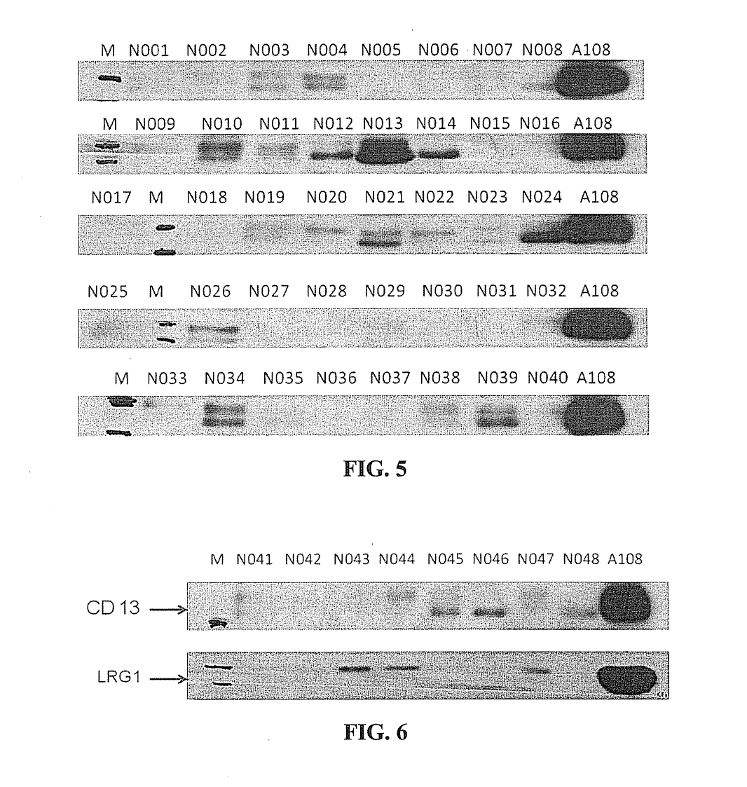

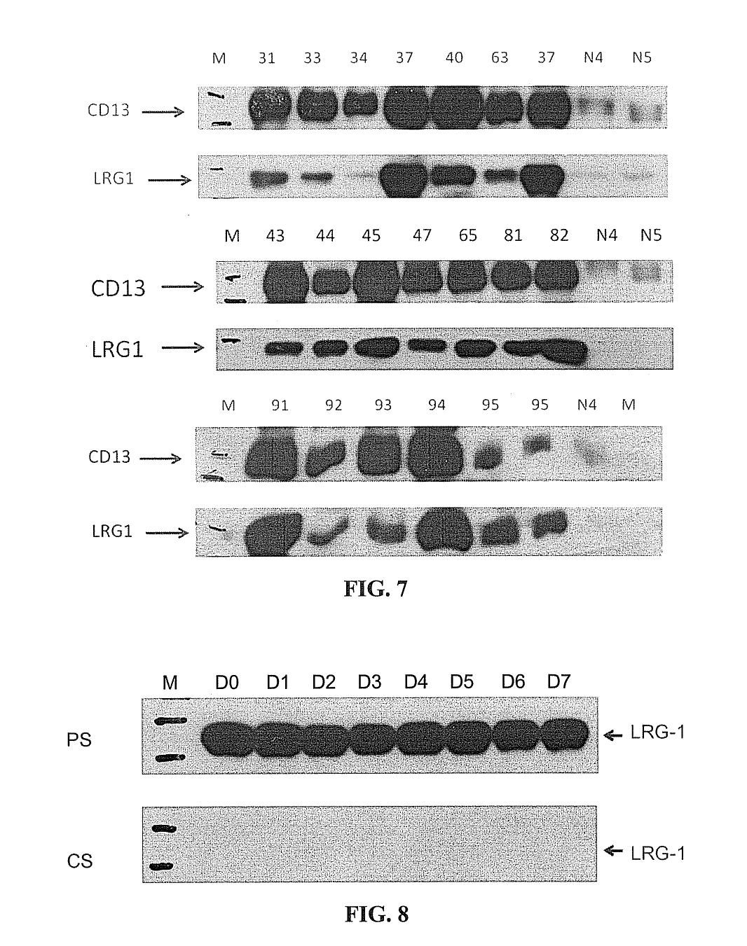

Plasma generated by healthy subjects has negligible levels of LRG1 (21-50 μg/mL) and CD13, which in many cases are almost undetectable by standard assays known in the art (FIGS. 4-7). In stark contrast to the low levels of LRG1 and CD13 found in healthy individuals are the highly elevated levels of both LRG1 and CD13 found in HCC positive individuals (FIGS. 4-7). In almost all cases, LC/MS and proteomic results show elevated levels of LRG1 (6/6) and CD13 (5/6) present in the urine of HCC positive subjects. In contrast to these results were the negative results for the control group, revealing elevated levels of LRG1 (0/6) and CD13 (1/6) present in the urine of HCC negative subjects. Elevated levels of LRG1 (45/48) and CD13 (48/48) have further been confirmed in second cohort of HCC positive patients with tandem mass spectrometry followed by western blot analysis. Additional control experiments have been conducted on a second cohort comprised of 54 apparently healthy individuals revealing increased levels of LRG1 (3/54) and CD13 (2/54) in only a single subject.

In some embodiments, the biological sample is placed in storage for a time prior to determining the levels of LRG1, CD13, or metabolites thereof. Stability is therefore an important factor in the design of diagnostic methods. The inventors therefore determining the stability of LRG1 at room temperature. A urine sample obtained from a patient with HCC was kept at room temperature for 0-7 days, followed by a western blot analysis using a polyclonal antibody to LRG-1. As shown in FIG. 8, the LRG1 present in the urine was stable during storage.

In another embodiment, the levels of LRG1, CD13, or the combination thereof in a test subject's bodily fluid sample are compared to a predetermined value to determine the risk to the subject of developing or having cancer. In one embodiment, the levels of LRG1, CD13, or the combination thereof in a test subject's bodily fluid sample may then be compared to a predetermined value to determine the risk to the subject of developing or having cancers of the bladder, prostate, urinary tract, the ovaries, kidneys, and the liver. In some embodiments, the levels of LRG1, CD13, or the combination thereof in a test subject's bodily fluid sample may then be compared to a predetermined value to determine the risk to the subject of developing or having hepatocellular carcinoma (HCC).

Methods for Measuring Levels of LRG1 and/or CD13 and/or One or More Metabolites Thereof

The presence of LRG1 and CD13 or metabolites thereof may be determined by any of a variety of standard protein analytic methods known in the art. These methods include absorbance, protein assays (e.g., DC protein assay, non-interfering protein assay, BCA protein assay, and the Lowry assay), gel electrophoresis (e.g., SDS-PAGE gel purification), a protein immunoblot (e.g., western blot), chromatography (e.g., size exclusion chromatography, ion exchange chromatography, and affinity chromatography), precipitation, ultracentrifugation, enzyme-linked immunosorbent assays (ELISA), and other common techniques known to one of ordinary skill in the art. The devices to carry out protein analysis can be collectively referred to herein as protein detectors. For example, serum leucine-rich alpha-2-glycoprotein-1 binds cytochrome c and inhibits antibody detection of this apoptotic marker in enzyme-linked immunosorbent assay (Cummins, et al., Apoptosis, Vol. 11, No. 7, pp. 1121-1129 (2006)) and ELISA for human serum leucine-rich alpha-2-glycoprotein-1 employing cytochrome c as the capturing ligand (Weivoda et al., J. Immunol. Methods, Vol. 336, No. 1, pp. 22-29 (2008)).

The levels of LRG1 and CD13 in a subject can be measured using an analytic device, which is a machine including a detector capable of identifying proteins such as LRG1 and CD13 and substantial protein fragments thereof (i.e., LRG1 or CD13 metabolites). The analytic device may be a spectrometric device, such as a mass spectrometer, an ultraviolet spectrometer, or a nuclear magnetic resonance spectrometer. A spectrometer is a device that uses a spectroscopic technique to assess the concentration or amount of a given species in a medium such as a biological sample (e.g., a bodily fluid). The analytic device used to measure the levels of LRG1, CD13 or their metabolites can be either a portable or a stationary device. In addition to including equipment used for detecting LRG1, CD13 and their metabolites, the analytic device can also include additional equipment to provide purification (i.e., physical separation) of analytes prior to analysis. For example, if the analyte detector is a mass spectrometer, it may also include a high performance liquid chromatograph (HPLC) or gas chromatograph (GC) to purify the choline-related trimethylamine-containing compounds before their detection by mass spectrometry, or it may be preferable to purify the protein using gel electrophoresis.

As indicated herein, mass spectrometry-based methods can be used to assess levels of LRG1, CD13 and their metabolites in a biological sample. Mass spectrometers include an ionizing source (e.g., electrospray ionization), an analyzer to separate the ions formed in the ionization source according to their mass-to-charge (m/z) ratios, and a detector for the charged ions. In tandem mass spectrometry, two or more analyzers are included. Such methods are standard in the art and include, for example, HPLC with on-line electrospray ionization (ESI) and tandem mass spectrometry.

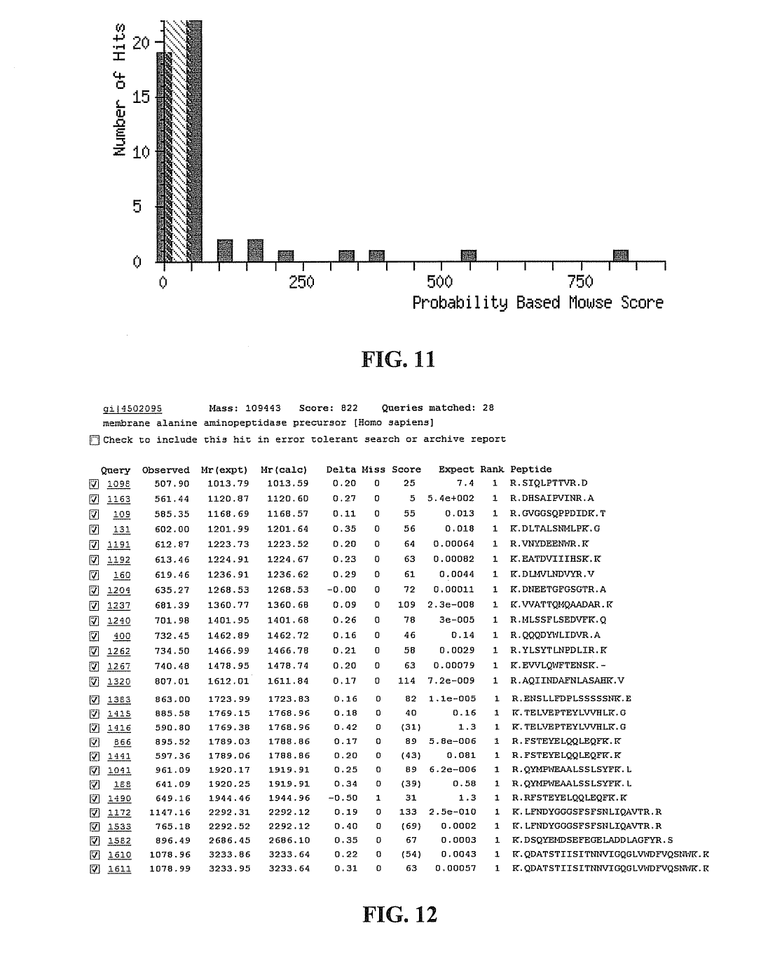

FIGS. 9-12 illustrate the use of mass spectrometry to identify mass spectrometry fragments resulting from the analysis of LRG1 and CD13. FIGS. 9 and 11 provides the results of a MOlecular Weight Search (Mowse) analysis, which is a method for identification of proteins from the molecular weight of peptides created by proteolytic digestion and measured with mass spectrometry. The probability-based MOWSE score formed the basis of development of Mascot, a proprietary software for protein identification from mass spectrometry data.

Mascot uses probability based scoring. This enables a simple rule to be used to judge whether a result is significant or not. Matches using mass values (either peptide masses or MS/MS fragment ion masses) are always handled on a probabilistic basis. The total score is the probability that the observed match is a random event. Reporting probabilities directly can be confusing. Partly because they encompass a very wide range of magnitudes, and also because a “high” score is a “low” probability, which can be ambiguous. For this reason, scores are reported as −10*LOG10(P), where P is the absolute probability. A probability of 10-20 thus becomes a score of 200.

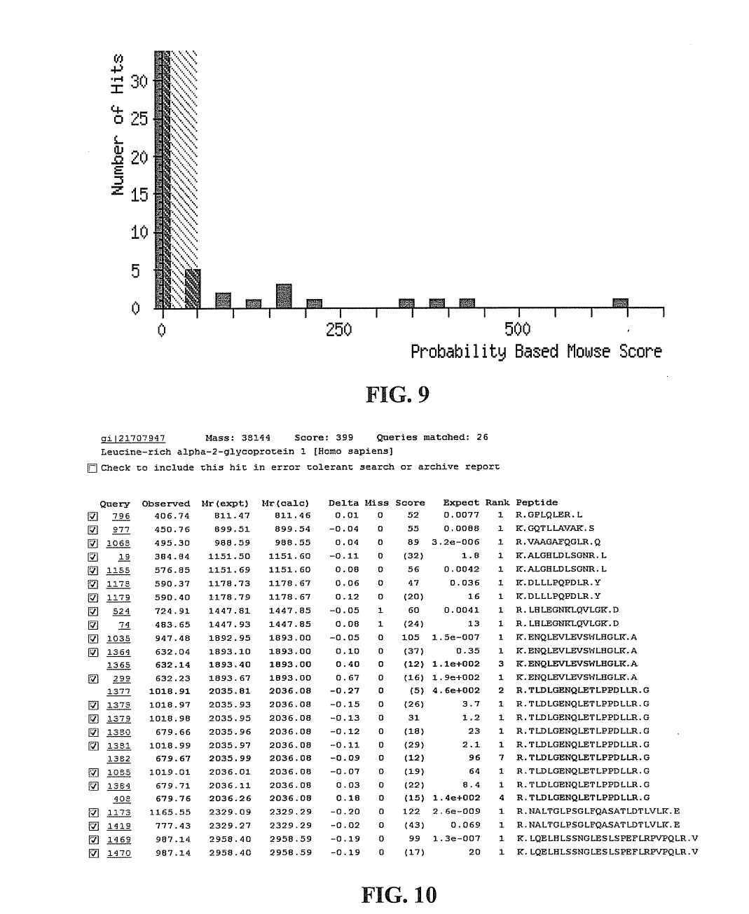

FIG. 10 shows sequence match results for the comparison of MS/MS fragments from patients to the amino acid sequence of human leucine-rich α-2-glycoprotein 1 (LRG1), while FIG. 12 shows the same type of data for CD13. This data shows the mass spectrometry amino acid fragments generated by mass spectrometry analysis that are useful for determining the levels of LRG1 and CD13.

Once the levels of LRG1, CD13 and/or their metabolites have been determined, they can be displayed in a variety of ways. For example, the levels of LRG1, CD13, and their metabolites can be displayed graphically on a display as numeric values or proportional bars (i.e., a bar graph) or any other display method known to those skilled in the art. The graphic display can provide a visual representation of the amount of LRG1 and/or CD13 in the biological sample being evaluated. In addition, in some embodiments, the analytic device can also be configured to display a comparison of the levels of LRG1 and/or CD13 in the subject's bodily fluid to a predetermined value based on levels of LRG1 and/or CD13 in comparable bodily fluids from a reference cohort.

### Predetermined Value

In some embodiments, the levels of LRG1, CD13, and the combination thereof in the bodily fluid sample obtained from a test subject is compared to a predetermined value. In one embodiment, the predetermined value is based upon the levels of LRG1, CD13, and the combination thereof in comparable samples obtained from the general population or from a select population of human subjects. For example, the select population may be comprised of apparently healthy subjects. “Apparently healthy,” as used herein, means individuals who have not previously had any signs or symptoms indicating the presence of HCC and other types of cancer, such as jaundice, bloating from ascites, easy bruising from blood clotting abnormalities, loss of appetite, unintentional weight loss, abdominal pain, nausea, emesis, or fatigue. Apparently healthy individuals also do not otherwise exhibit symptoms of disease including, alcoholism, hepatitis B or C, aflatoxin, cirrhosis, hemochromatosis, Wilson's disease, and Type 2 Diabetes. In other words, such individuals, if examined by a medical professional, would be characterized as healthy and free of symptoms of disease.

In one embodiment, the predetermined value is related to the value used to characterize the levels of LRG1, CD13, and the combination thereof in the bodily sample obtained from a test subject. Thus, if the levels of LRG1, CD13, and the combination thereof are an absolute value such as the units of LRG1, CD13, and the combination thereof per milliliter of urine, the predetermined value is also based upon the units of LRG1, CD13, and the combination thereof per ml of urine in individuals in the general population or a select population of human subjects. Similarly, if the levels of LRG1, CD13, and the combination thereof are a representative value such as an arbitrary unit obtained from a cytogram, the predetermined value is also based on the representative value.

The predetermined value may take a variety of forms. In one embodiment, the predetermined value may be a single cut-off value, such as a median or mean. In some embodiments, the predetermined value may be established based upon comparative groups such as where risk in one defined group is double the risk of another defined group. In another embodiment, the predetermined value may be a range, for example, where the general population is divided equally (or unequally) into groups, such as a low risk group, a medium risk group and a high-risk group, or into quadrants, the lowest quadrant being individuals with the lowest risk the highest quadrant being individuals with the highest risk.

The predetermined value may be derived by determining the level of LRG1 or CD13 in the general population. Alternatively, the predetermined value can be derived by determining the level of LRG1 or CD13 in a select population, such as an apparently healthy, non-alcohol consuming population. For example, an apparently healthy, non-alcohol consuming population may have a different normal level of LRG1 or CD13 than will an alcohol consuming population or a population whose members have had a prior liver disorder. Accordingly, the predetermined values selected may take into account the category in which an individual falls. Appropriate ranges and categories may be selected with no more than routine experimentation by those of ordinary skill in the art.

In some embodiments, predetermined values for the levels of LRG1, CD13, or both, for example, mean levels, median levels, or “cut-off” levels, are established by assaying a large sample of individuals in the general population or a select population and using a statistical model such as the predictive value method for selecting a positivity criterion or receiver operator characteristic curve that defines optimum specificity (highest true negative rate) and sensitivity (highest true positive rate). A “cutoff” value may be determined for the presence of LRG1, CD13, or the combination thereof.

Comparison of LRG1 and CD13 Levels in the Bodily Sample from the Test Subject to the Predetermined Value

The levels of each risk predictor (i.e., LRG1, CD13, or both) in a test subject's bodily sample may be compared to a single predetermined value or to a range of predetermined values. If the level of the present risk predictor in the test subject's bodily sample is greater than the predetermined value or range of predetermined values, the test subject is at greater risk of developing or having liver cancer than individuals with levels comparable to or below the predetermined value or predetermined range of values. In contrast, if the level of the present risk predictor in the test subject's bodily sample is below the predetermined value or range of predetermined range, the test subject is at a lower risk of developing or having liver cancer than individuals with levels comparable to or above the predetermined value or range of predetermined values.

The comparison can be conducted by any suitable method known to those skilled in the art. For example, the comparison can be carried out mathematically or qualitatively by an individual operating the analytic device or by another individual who has access to the data provided by the analytic device. Alternately, the steps of determining and comparing the levels of LRG1, CD13, or their metabolites can be carried out electronically (e.g., by an electronic data processor).

For example, a test subject who has a higher level of LRG1, CD13, or a combination thereof as compared to the predetermined value is at high risk of developing HCC, and a test subject who has a lower level of LRG1, CD13, or a combination thereof as compared to the predetermined value is at low risk of developing HCC. The extent of the difference between the test subject's risk predictor levels and predetermined value is also useful for characterizing the extent of the risk and thereby, determining which test subject's would most greatly benefit from certain aggressive therapies. In those cases, wherein the predetermined value ranges are divided into a plurality of groups, such as the predetermined value ranges for individuals at high risk, average risk, and low risk, the comparison involves determining into which group a test subject's level of the relevant risk predictor falls.

The method can also include the step of providing a report indicating the subject is in need of cancer therapy if levels of LRG1 and/or CD13 or one or more metabolites thereof are higher than at least one of the one or more predetermined values. For example, the apparatus for carrying out the method can include a processor coupled to the protein detector and adapted to quantify the data representing the signals from the detector, and adapted to perform the multivariate statistical analysis, compare the output value to the first reference value and the second reference value, and calculate the risk score; and an output display coupled to the processor and configured to report the risk score.

The present diagnostic tests may be useful for determining if and when therapeutic agents which are targeted at preventing liver cancer should and should not be prescribed for a test subject. For example, test subjects with values of LRG1 or CD13 (μg/mL urine) above a certain cutoff value, or that are in the higher tertile or quartile of a “normal range,” could be identified as those in need of more aggressive intervention.

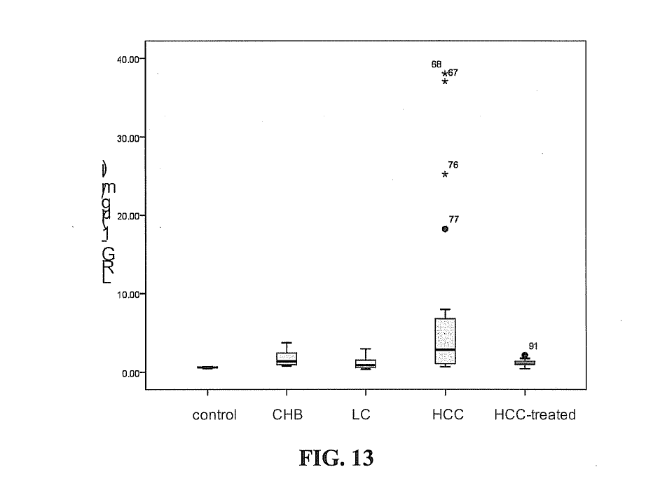

One of the most attractive findings of increased levels of LRG1 and CD13 as a predictor of risk for HCC is that it may represent an independent marker to identify individuals with increased risk for HCC and other cancers including, bladder, prostate, urinary tract, the female reproductive system, kidney, and other cancers of the liver. Thus, the present diagnostic tests are especially useful to identify individuals at increased risk who might otherwise not have been identified by existing screening protocols/methods. Moreover, the present risk predictors can be used in combination with currently used risk predictors, such as α-fetoprotein levels, and algorithms based thereon to more accurately characterize a test subject's risk of developing or having cancer. The ability of the method to distinguish HCC from other liver diseases is shown in FIG. 13. FIG. 13 shows the results of a determination of LRG1 levels in the urine of patients with HCC (31 cases) and HCC tumors removed surgically (18 cases), chronic heptatitis (14 cases) and liver cirrhosis (20 cases) as evaluated by ELISA. The results clearly distinguish patients having HCC from patients who previously had HCC but have been successfully treated and patients having other types of liver disease.

In another embodiment, the present invention relates to kits that include reagents for assessing levels of one or more of LRG1, CD13, or metabolites thereof in biological samples obtained from a test subject. Such assays have appropriate sensitivity with respect to predetermined values selected on the basis of the present diagnostic tests. In certain embodiments, the kits also include printed materials such as instructions for practicing the present methods, or information useful for assessing a test subject's risk of having cancer. Examples of such information include, but are not limited to cut-off values, sensitivities at particular cut-off values, as well as other printed material for characterizing risk based upon the outcome of the assay. In some embodiments, such kits may also comprise control reagents, e.g. known amounts of LRG1, CD13, or metabolites thereof.

### Evaluation of Cancer Therapeutic Agents

The present diagnostic tests are also useful for evaluating the effect of chemotherapeutic agents on test subjects who have been diagnosed as having or as being at risk of developing cancer. An additional aspect of the invention provides a method for evaluating the effect of cancer therapy in a subject suspected of having or diagnosed as having cancer. Examples of cancer therapy include, surgery to remove a portion of the liver, freezing cancer cells, localized heat to destroy cancer cells, chemotherapy, radiation therapy, and targeted drug therapy (e.g., Sorafenib or Nexavar). In particular, the method is suitable for evaluating the effect of therapy of liver cancer or hepatocellular carcinoma.

The method includes determining levels of LRG1 and/or CD13 or one or more metabolites thereof in a biological sample taken from the subject prior to therapy and determining levels of LRG1 and/or CD13 or one or more metabolites thereof in a corresponding biological sample taken from the subject during or following therapy, wherein a decrease in levels of LRG1 and/or CD13 or one or more metabolites thereof in the sample taken after or during therapy as compared to levels of LRG1 and/or CD13 or one or more metabolites thereof in the sample taken before therapy is indicative of a positive effect of the therapy on cancer in the treated subject.

In specific embodiments, the level of LRG1, the level of CD13, or the levels of one or more metabolites of LRG1 or CD13 in the biological sample can determined. Suitable biological samples include those described herein, such as urine.

The levels of LRG1 and/or CD13 or one or more metabolites thereof in the method of evaluating cancer therapy can be determined using any suitable protein analytic method. Examples of protein analytic methods are described herein, and include immunoassay and protein separation following by protein analysis using mass spectrometry

Such evaluation comprises determining the levels of one or more of the present risk predictors including LRG1, CD13, or a combination thereof, in a bodily sample taken from a test subject prior to administration of the therapeutic agent and a corresponding bodily fluid taken from a test subject following administration of the therapeutic agent. A decrease in the level of the selected risk factor in the sample taken after administration of the therapeutic as compared to the level of the selected risk factor in the sample taken before administration of the therapeutic agent is indicative of a positive effect of the therapeutic agent on cancer in the treated subject.

The following example is included for purposes of illustration and is not intended to limit the scope of the invention.

### EXAMPLE

### Example 1

### Method of Determining LRG1 and CD13 Levels

### General Procedures

Human leucine-rich α-2-glycoprotein-1 (LRG1) and aminopeptidase N (CD13) were isolated and quantified. Proteins in the urine were separated by 10% sodium dodecyl sulfate polyacrylamide gel electrophoresis (SDS-PAGE) and the protein bands were excised. The proteins in the gel slice were subjected to reduction and alkylation of cysteines in 50% 0.1 M ammonium carbonate, 48.75% acetonitrile, 1% idoethanol, and 0.25% triethylphosphone for 1 hour (final pH 10), followed by digestion of trypsin in buffer (50 mM NH₄HCO₃, pH 8.0) overnight. The tryptic peptides were extracted by 5% formic acid, 50% Acetonitrile and then condensed to less than 10 μL by speed vacuum, and subjected to be analyzed by LC-MS/MS (LC/MS R-P column, 100×0.300 mm, 5 μM/300 Å, Polymeric C18; MS Model Bruker Ion-Trap). The tandem MS data were searched by using the MASCOT database (Matrix Science).

### Preparation of Bodily Fluid Sample

The first urine in the morning was collected with a clean disposable plastic container. The urine sample (10 ml) was stored in a 10 ml centrifuge tube marked with the patient information at −80° C. until analysis. If the urine was turbid, it would be centrifuged at 1,500 rpm for 10 minutes to remove precipitates before storage. To avoid contamination by bacteria from the genital tract and urethra, the midstream of urine was collected.

### Mass Spectrometry

LC/ESI/MS/MS was used to identify peptide fragments resulting from the trypsin digest of proteins isolated from SDS-PAGE gels. Sample preparation: After reduction and alkylation of cysteines (with 50% 0.1 M ammonium carbonate, 48.75% acetonitrile, 1% idoethanol, and 0.25% triethylphosphone for 1 hour (final pH 10)), samples were digested with trypsin in 50 mM ammonium bicarbonate for overnight. Peptides were extracted with 5% formic acid, 50% acetonitrile and then condensed to less than 10 μL by speed vacuum. After reconstituting with loading buffer (0.1% formic acid, 2% acetonitrile), samples were manually injected to a cartridge with a flow rate of 10 μL 0.1% formic acid 2% acetonitrile. Then the peptides were eluted with a gradient from 1% to 60% mobile phase B within 2 hours (mobile phase A: 0.1% formic acid, 2% acetonitrile in deionized water; mobile phase B: 0.1% formic acid in acetonitrile). Analyses were performed using electrospray ionization in positive-ion mode. Nitrogen (N₂) was used as the curtain gas in the electrospray interface.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of characterizing a subject's risk of having cancer, comprising the steps of:
(a) determining a level of LRG1 and/or CD13 or one or more metabolites thereof in a biological sample obtained from the subject by analyzing the biological sample using a protein analytic method; and
(b) comparing the determined level of the LRG1 and/or CD13 or one or more metabolites thereof in the biological sample to at least one predetermined value;
wherein a subject that has an elevated level of at least one of the LRG1 and/or CD13 or one or more metabolites thereof in comparison with one or more of the predetermined values is at risk of having cancer.

2. The method of claim 1, further comprising providing a report indicating the subject is in need of cancer therapy if levels of LRG1 and/or CD13 or one or more metabolites thereof are higher than at least one of the one or more predetermined values.

3. The method of claim 1, wherein the level of LRG1 in the biological sample is determined.

4. The method of claim 1, wherein the level of CD13 in the biological sample is determined.

5. The method of claim 1, wherein the biological sample is whole blood, serum, plasma, urine, cerebrospinal fluid, or bronchoalveolar lavage.

6. The method of claim 1, wherein the biological sample is urine.

7. The method of claim 1, wherein the cancer is selected from the group consisting of cancers of the liver, bladder, prostate, urinary tract, ovaries, or kidney.

8. The method of claim 1, wherein the cancer is hepatocellular carcinoma.

9. The method of claim 1, wherein the subject is human.

10. The method of claim 1, wherein the protein analytic method is an immunoassay.

11. The method of claim 1, wherein the protein analytic method includes the steps of protein separation and protein analysis by mass spectrometry.

12. The method of claim 1, wherein the determining and comparing are carried out electronically.

13. The method of claim 1, wherein the biological sample is analyzed in vitro.

14. A method for evaluating the effect of cancer therapy in a subject suspected of having or diagnosed as having cancer, comprising: determining levels of LRG1 and/or CD13 or one or more metabolites thereof in a biological sample taken from the subject prior to therapy and determining levels of LRG1 and/or CD13 or one or more metabolites thereof in a corresponding biological sample taken from the subject during or following therapy, wherein a decrease in levels of LRG1 and/or CD13 or one or more metabolites thereof in the sample taken after or during therapy as compared to levels of LRG1 and/or CD13 or one or more metabolites thereof in the sample taken before therapy is indicative of a positive effect of the therapy on cancer in the treated subject, and wherein any step in which the levels of LRG1 and/or CD13 or one or more metabolites thereof are determined is carried out using a protein analytic method.

15. The method of claim 14, wherein the level of LRG1 in the biological sample is determined.

16. The method of claim 14, wherein the level of CD13 in the biological sample is determined.

17. The method of claim 14, wherein the biological sample is urine.

18. The method of claim 14, wherein the cancer is hepatocellular carcinoma.

19. The method of claim 14, wherein the protein analytic method is an immunoassay.

20. The method of claim 14, wherein the protein analytic method includes the steps of protein separation and protein analysis by mass spectrometry.
